(19) United States
(12) Patent Application Publication
(10) Pub. No.: US 2025/0186184 A1
(43) Pub. Date: Jun. 12, 2025

(54) SOCKET VALVE

(71) Applicant: OTTOBOCK SE & CO. KGAA, Duderstadt (DE)

(72) Inventors: René KUNZE, Duderstadt (DE); André MÜLLER, Duderstadt (DE); Titus SCHINKEL, Duderstadt (DE); David BRAND, Duderstadt (DE); Michael DE BORTOLI, Duderstadt (DE)

(21) Appl. No.: 18/963,311

(22) Filed: Nov. 27, 2024

(30) Foreign Application Priority Data
Dec. 6, 2023 (DE) 102023134136.7

(51) Int. Cl. A61F 2/00; A61F 2/80

(52) U.S. Cl. CPC A61F 2/00; A61F 2002/805

(57) ABSTRACT

The invention relates a socket valve for ventilating and venting a prosthesis socket with a base body (10), the base body (10) has a through bore (12), a seal carrier (30) mounted on the base body (10) so as to be rotatable about an axis of rotation (5) is attached to the base body (10), which is in fluidic connection with the through-bore (12) and closes or opens a ventilation duct (14) which is in fluidic connection with the through-bore (12), wherein the closable ventilation duct (14) is formed in the base body (10) and is sealed off from the through bore (12) via a seal (50) arranged between the seal carrier (30) and the base body (10) in a first position of the seal carrier (30) and is in fluidic connection with the through bore (12) in an unsealed manner in a second position of the seal carrier (30), and the seal (50) is positioned in an inclined manner with respect to the axis of rotation (5).

### CROSS REFERENCE TO RELATED APPLICATION

This is a application claims the benefit of German Patent Application No. 102023134136.7, filed 6 Dec. 2023, the disclosure of which is incorporated herein, in its entirety, by this reference.

### TECHNICAL FIELD

The invention relates to a socket valve for venting and ventilating a prosthesis socket with a base body which has a through bore and to which is attached a seal carrier which is mounted on the base body so as to be rotatable about an axis of rotation, the seal carrier is in fluidic connection with the through bore and closes or opens a ventilation duct which is in fluidic connection with the through bore. In an advantageous embodiment, the socket valve is designed as a pressure-activated ejection valve.

### BACKGROUND

Prostheses replace limbs that do not exist or no longer exist via functional components that can vary in complexity. Prostheses can be positioned on upper and lower limbs and are attached to the limb in various ways. Initially, prostheses, especially lower limb prostheses, were attached to the trunk or limb using straps, bands or flexible tensioning devices. A prosthesis socket is used to hold the limb stump, which has fastening devices for the actual prosthesis component, e.g. a prosthesis knee joint and/or a prosthesis foot or is also formed in one piece with such a prosthesis component. In addition to fastening via a fastening element fixed in a bone, the vast majority of prosthetic fittings are made using a prosthetic socket, which forms a receiving space for the limb stump. A so-called liner, which consists of a flexible and elastic plastic, for example silicone or another polymer or with such a component, is arranged between the dimensionally stable prosthetic socket and the limb stump. The inside of the liner adheres to the skin surface. In addition to mechanical locking via a so-called pin-lock system or in addition to this, the prosthesis socket is attached to the liner using so-called suction socket technology, in which the prosthesis socket is cup-shaped and sealed airtight against the liner. When the liner is inserted together with the residual limb into the receiving space of the prosthesis socket, the air inside is pressed out via an ejection valve. If the receiving space is completely or almost completely empty and the seal is sufficiently tight, the liner is effectively secured within the prosthesis socket in this way. Alternatively, the socket can be worn without a liner. These so-called full-contact sockets may have a silicone inner socket, but variants without an inner socket are also known.

In order to be able to release the connection between the prosthesis socket and the liner, it must be possible to equalize the pressure between the environment and the space between the prosthesis socket and the liner. This can be achieved via a separate inlet valve or an ejection valve that can be switched between two positions. When walking, air is forced out of the prosthesis socket below a seal between the prosthesis socket and the liner during a loading phase. This continuously provides sufficient negative pressure to ensure that the prosthesis socket is held securely on the liner. The repeated expulsion of air through the expulsion valve is necessary to remove any air that may enter due to leaks during use.

The introduction of air into the space between the liner and the prosthetic socket below a seal and a sealing lip is necessary to allow the user to get out of the socket. U.S. Pat. No. 2,790,180 A discloses an artificial limb with a prosthetic socket with a recess for a substantially airtight accommodation of a limb stump with a plurality of outlet openings between the interior of the socket and the environment. Each of these outlet ducts has a different flow resistance and a valve to open or close a selected duct. A bore and a valve seat are formed in a screwed-in valve, in which a spring-loaded valve body with a longitudinal bore is arranged. The valve body in the valve seat closes a transverse bore, which is in fluidic connection with the longitudinal bore in the open state. The valve body can be rotated to different positions in the valve seat in order to set different flow resistances or a closed position.

U.S. Pat. No. 7,993,413 B 2 relates to a valve system for prostheses with a dimensionally stable prosthesis socket, in the distal area of which an ejection valve is arranged. The ejection valve has a spring-loaded valve body which is pressed into a valve seat which is in fluidic connection with the interior of the prosthesis socket. As soon as the force on the valve body is greater than the spring force due to the air pressure inside the prosthesis socket, the valve body is lifted out of the valve seat and air can escape. The valve seat can be moved to different positions so that ambient air can flow into the prosthesis socket in an open position.

U.S. Pat. No. 9,615,946 B2 relates to a prosthesis device with a prosthesis socket and a prosthesis foot attached thereto with two plates that can move relative to one another. A vacuum pump is driven by at least one relative movement of the two plates and sucks air out of the prosthesis socket. A valve is arranged on the socket, which enables ejection, a vacuum bypass and ventilation.

### SUMMARY

The object of the present invention is to provide a socket valve with a venting function and a venting function, which has a compact design and requires no or only a small height stroke for venting the valve.

This task is solved by a socket valve with the features of the main claim. Advantageous embodiments and further embodiments of the invention are disclosed in the subclaims, the description and the figures.

The socket valve for ventilating and venting a prosthesis socket with a base body which has a through bore and to which is attached a seal carrier which is mounted on the base body so as to be rotatable about an axis of rotation, is in fluidic connection with the through bore and closes or opens a ventilation duct which is in fluidic connection with the through bore, characterized in that the closable ventilation duct is formed in the base body and is sealed off from the through bore via a seal arranged between the seal carrier and the base body, that the closable ventilation duct is formed in the base body, which is sealed off from the through-hole via a seal arranged between the seal carrier and the base body in a first position of the seal carrier and is in fluidic connection with the through-hole in an unsealed manner in a second position of the seal carrier, the seal being positioned inclined to the axis of rotation of the seal carrier. In particular, the seal is arranged in a plane that is inclined to the axis of rotation so that, for example, an O-ring can be installed in a straight line in one plane and a gap between the through bore and the seal carrier can be easily sealed. The seal, which is oriented at an angle to the axis of rotation of the seal carrier, reduces the overall installation height while maintaining a simple design. The seal can be arranged statically within the base body; alternatively, the seal is arranged on the seal carrier in a corresponding receptacle or seal groove and rotates together with the seal carrier about the axis of rotation relative to the base body. In a first position of the seal carrier, the through-hole is sealed off from the closable ventilation duct so that no air from outside can enter the prosthesis shaft through the ventilation duct and the through-hole.

In one embodiment, an outlet duct is formed within the seal carrier, which is in fluidic connection with the through bore, whereby a valve body is arranged within the outlet duct. The valve body within the seal carrier advantageously closes the through bore, in particular with respect to a differential pressure between the prosthesis socket and the environment, in such a way that air can escape when the pressure within the prosthesis socket is greater than the external pressure, but conversely air is prevented from flowing through the valve body into the prosthesis socket.

In one embodiment and further development, the seal body is mounted on the base body without axial stroke along the axis of rotation, so that when the seal carrier is rotated relative to the base body, the seal carrier does not move away from the base body and protrudes further. As a result, the overall height is kept constant in the various positions and is also reduced compared to a thread-based bearing.

In one embodiment and further development of the socket valve as an ejection valve, the valve body is arranged in a valve seat, which is formed in particular on the seal carrier. The valve body is held pretensioned in a closed position by a valve spring so that air only escapes from the prosthesis socket through the socket valve, which is then designed as an ejection valve, when the differential pressure exceeds a threshold value. The valve spring ensures that the valve body is moved back into the valve seat after the excess pressure has ceased and the through bore is sealed, regardless of the position of the seal carrier relative to the base body. Alternatively, the valve body is designed as a beak valve or diaphragm valve, which has an inherent restoring force or forms a non-return valve due to its elastic structure. The valve body can therefore be designed as any type of non-return valve. It is essential that the valve body opens when there is a differential pressure and works as an ejection valve and, conversely, prevents air from entering through the through bore and the outlet duct.

In one embodiment, the seal carrier is mounted on the base body via at least one bearing body, with the bearing body running along in a bearing groove during rotation. The bearing groove ensures the allocation of the bearing body during rotation and in the individual positions of the seal carrier relative to the base body, with the bearing groove being oriented in particular in a plane perpendicular or essentially perpendicular to the axis of rotation. The orientation of the bearing groove perpendicular to the axis of rotation ensures that the seal carrier does not move in the axial direction relative to the base body during adjustment from the first position to the second position. The bearing groove does not have to have a constant radius over the circumference, but can vary in its circumference. In particular, it is advantageous if the radius of the bearing groove is smallest in the closed state when the bearing groove is formed in the seal carrier. As a result, a preload is achieved between the seal carrier and the base body when it is rotated into the first, closed position and the closed position is secured. If the bearing groove is formed within the base body, the radius is preferably largest in the first position.

In a further development, the bearing body is resiliently mounted in the base body, which on the one hand facilitates assembly and on the other hand ensures a durable and low-wear mating of the seal carrier and base body. The resilient mounting of the bearing body or the bearing body in the base body leads to an increased contact pressure of the bearing body in the bearing groove, particularly when the radius is changed. The bearing body or bearing bodies are preferably arranged in the base body within a recess or bore that is oriented essentially radially outwards and in which the bearing body can be inserted. A resilient mounting makes it possible to press the bearing body radially inwards during assembly and to move the seal carrier over the bearing bodies. If the bearing bodies or the bearing body are located on the bearing groove, the bearing bodies are pressed into the bearing groove by the resilient mounting. Conversely, it is possible to arrange the bearing body elastically within the seal carrier. If the seal carrier is designed as a cap that is arranged over the base body, the bearing inside the base body is simpler due to the available space.

In a further development, at least one detent recess for the bearing body is formed in the bearing groove. In particular, the detent recess ensures that the seal carrier is held in the first, closed position. In addition, the detent recess provides haptic feedback and a noticeable increase in the force required to move the seal carrier from the first position to the second position. Several detent recesses can be provided for several bearing bodies. The detent recesses can be oriented in such a way that the respective bearing bodies engage in the first position, in particular all bearing bodies engage. Detent recesses can also be provided for the second, open position. Furthermore, detent recesses can be present in the position in which the base body and the seal carrier can be separated from each other.

In one embodiment, the seal carrier has a wall that at least partially surrounds the base body on the outside with a projection that projects radially inwards. The inwardly projecting projection can form an undercut so that a form-fit connection with the base body can be achieved with simultaneous rotatability. The inwardly projecting projection engages behind a lower edge or a groove within the base body and thus fixes the seal carrier axially to the base body. The projection can, for example, be formed or arranged on radially outwardly resilient wall sections. In the case of a closed, circumferential wall, at least one recess is formed in the projection, through which at least one of the bearing bodies can be passed for mounting. After mounting, the seal carrier is twisted so that the bearing body is additionally secured.

In one embodiment, the number of bearing bodies corresponds to the number of recesses. Advantageously, the size of the bearing bodies corresponds to the size of the recesses, so that the seal carrier can only be mounted on the base body if all the recesses are aligned to correspond to the respective bearing body. Advantageously, mounting is only possible in exactly one position of the seal carrier relative to the base body with radially resilient, axially essentially stationary bearing bodies. The corresponding dimensions of the bearing body and recesses improve the allocation of the components to each other and ensure precise assembly.

In one embodiment, a stop is formed between the base body and the seal carrier to limit the angle of rotation about the axis of rotation of the seal carrier relative to the base body. The stop is designed, for example, as a pin, bolt or projection, in particular as a resiliently mounted bolt or projection, and engages in a groove within the seal carrier or the base body. Due to the limited space available, the stop is arranged in the base body in particular, while a corresponding groove or another positive locking element is arranged or formed in or on the seal carrier.

In one embodiment, a seal is arranged between the valve body and the valve seat in order to prevent air from flowing back through the outlet duct into the prosthesis shaft.

For mounting the socket valve, it is provided in a further development that the base body has an external thread surrounding the through bore so that the base body can be screwed into the prosthesis socket or screwed onto it. In one embodiment, the base body has a form-locking element, for example an internal hexagon, a polygonal toothing, a slot, an internal polygon or external polygon or the like, for engaging with an assembly tool. This makes it easier to fit the base body to the prosthesis socket.

In one embodiment, the base body is inserted into a base with a pump connection so that the socket valve can be connected to other components for adjusting the internal pressure within the prosthesis socket. Alternatively or additionally, a fastening device for a suction connection can be arranged on the seal carrier, whereby the fastening device can be a thread, a carabiner connection or a clamp connection, for example. The suction connection is arranged on the seal carrier and, in a design with a valve body, behind the valve body in the direction of flow, so that the air is sucked out through the ventilation or outlet duct when a vacuum is applied or when air is sucked out of the prosthesis socket. In the design with a pump connection on the base, this pump connection is formed in front of the valve body or non-return valve or ventilation duct in the direction of flow and has a fluidic connection to the through bore.

To ensure a stable position of the seal with a movable mounting when the seal can be rotated relative to the base body, in one embodiment a sealing groove is formed in an outer side of a journal of the seal carrier, in which the seal is mounted. Alternatively, the sealing groove is formed in the base body and the seal slides on the seal body. The seal can be provided with a friction-reducing coating, for example a PTFE coating or a CVD coating, for example Parylene. The pin is inserted into the through hole and is sealed via the seal.

In a further development a silencer is arranged in the through bore of the base body, for example on the valve body.

All embodiments and further developments of the socket valve can be used in combination with each other individually or together, provided they are not technically mutually exclusive.

### BRIEF DESCRIPTION OF THE DRAWINGS

In the following, embodiments of the invention are explained in more detail with reference to the figures. Identical reference signs denote identical components; not all reference signs are shown in all figures in order to ensure clarity and comprehensibility. The figures show

FIG. 1—a sectional view of a socket valve in two positions;

FIG. 2—another sectional view of a socket valve as shown in FIG. 1;

FIG. 3—an exploded view of the socket valve;

FIG. 4—a horizontal section through the socket valve in different positions;

FIG. 5—a horizontal section in another plane;

FIG. 6—a variant with a base and a pump connection in sectional view;

FIG. 7—a perspective view of the embodiment shown in FIG. 6;

FIG. 8—perspective view of a variant with a suction connection;

FIG. 9 is a cross-sectional view of FIG. 8;

FIG. 10—an illustration of FIG. 9 in a non-assembled state;

FIGS. 11 to 13—a variant in cross-section;

FIG. 14—a horizontal section;

FIG. 15—another variant in vertical section;

FIG. 16—bottom views in different positions;

FIG. 17—a variant in vertical section;

FIG. 18—a valve body as shown in FIG. 17 in perspective view;

FIG. 19—a perspective view of a seal carrier from below;

FIG. 20—a base body in perspective view;

FIG. 21—a variant of a seal carrier in cross-sectional view;

FIG. 22—perspective view of the seal carrier shown in FIG. 21;

FIG. 23—a variant of a valve body;

FIGS. 24 and 25—Sectional views of a socket valve; and

FIG. 26—a variant of a socket valve.

### DETAILED DESCRIPTION

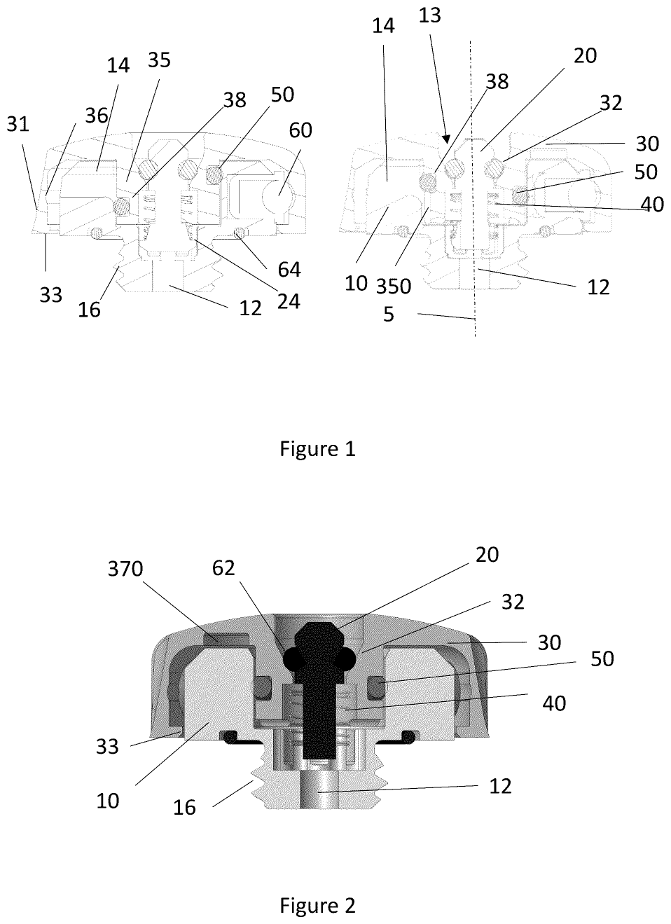

FIG. 1 shows a cross-sectional view of a socket valve for ventilating and deaerating a prosthesis socket in the form of an ejection valve in two positions. In the left-hand position of FIG. 1, the socket valve is shown in a first position with a sealed ventilation duct 14 and in the right-hand position with an open ventilation duct 14. The socket valve has a base body 10 which, in the illustrated embodiment example, has a disk-shaped head with a downwardly projecting spigot on which an external thread 16 is formed. A through bore 12 is formed within the base body 10 and the pin and is arranged centrally. A seal 64 is arranged in a groove on the underside of the head of the base body 10, which serves to provide a seal between the socket valve and a prosthesis socket or a prosthesis socket component when the base body is screwed in. Within the base body 10, a central pin 35 of a seal carrier 30 is arranged in the through bore 12, which is mounted rotatably about an axis of rotation 5, which in the embodiment example shown is also the central axis of the base body 10. The seal carrier 30 is designed as a cap, which has a cover and a side wall 31 that circumferentially surrounds the base body 10 in the embodiment example shown. An inwardly projecting projection 33 is formed on the underside of the side wall 31. A bearing groove 36 for the bearing body 60 is formed on the inside of the circumferential side wall 31. In the illustrated embodiment example, the bearing groove 36 extends in a plane substantially perpendicular to the axis of rotation 5 of the seal carrier 30. The seal carrier 30 is held in the bearing groove 36 by the bearing bodies 60, which are guided in the bearing groove 36 for example as rolling balls or sliding ball segments and moved within the plane of the bearing groove 36. With a vertical or almost vertical orientation, there is no axial stroke in the direction of the axis of rotation 5 when the seal carrier 30 rotates.

A valve seat 32 for a valve body 20 is formed within the seal carrier 30, which is conical in the embodiment example shown. The valve body 20 is sealed inside the valve seat 32 via a seal 62, which is provided with the reference sign in FIG. 2. A valve spring 40 loads the valve body 20 in the direction of a closed position, in which the valve body 20 with the seal 62 is pressed against the valve seat 32. The valve body 20 as well as the valve spring 40 is arranged in an outlet duct 13 within the seal carrier 30. The outlet duct 13 extends through the seal carrier 30 and the pin 35 and is in fluidic connection with the through bore 12 of the base body 10. It establishes a fluidic connection between the interior of a prosthesis socket and the open environment when the ejection valve is screwed in. The fluidic connection is sealed with a valve body 20 pressed against the valve seat 32 when the ejection valve is closed. This is achieved by the valve spring 40 until the internal pressure within the prosthesis shaft and thus within the through bore 12 and the outlet duct 13 is greater than the ambient pressure or the closing force of the valve spring 40. As soon as this threshold value is exceeded, the pressurized air lifts the valve body 20 out of the valve seat 32 and can escape from the prosthesis socket. Conversely, if the internal pressure is less than the contact pressure of the valve spring 40 or less than the ambient pressure, the valve body 20 is moved into the valve seat 32 so that no external air from the environment can enter the prosthesis socket. It is therefore a spring-loaded non-return valve. The valve spring 40 is supported on the one hand on a shoulder inside the outlet duct 13 and on the other hand on a support 24 on the valve body 20.

The ventilation duct 14 is formed within the base body 10, which in the illustrated embodiment example is formed as a channel-shaped depression that leads radially outwards from the through-hole 12. The ventilation duct 14 is formed on the upper side of the base body 10 and is sealed by a seal 50 in the first position of the seal carrier 30 relative to the base body 10, as shown in the left-hand illustration. In the embodiment shown, the seal 50 is arranged in a sealing groove 38, which is formed on the pin 35 of the seal carrier 30. The outer diameter of the pin 35 essentially corresponds to the inner diameter of the through-hole 12 in the area in which the pin 35 is inserted into the through-hole 12. At least one duct 350 is formed on the outside of the pin 35, which extends longitudinally along the outside of the pin 35 and can establish a fluidic connection between the through-hole 12 and the ventilation duct 14. The established fluidic connection between the through-hole 12 and the ventilation duct 14 is shown in the right-hand representation of FIG. 1. The channel 350 on the outside of the pin 35 has been brought into an overlapping alignment with the ventilation duct 14, so that air can flow unhindered through the ventilation duct 14 below the seal 15 through the channel 350 into the through-hole 12 and thus equalize the pressure between the environment and the interior of the prosthesis socket.

In the left-hand illustration in FIG. 1, the seal carrier 30 is rotated in such a way that the seal 50 seals the ventilation duct 14. This is achieved by positioning the seal 50 at an angle to the axis of rotation 5. In the illustrated embodiment example, the seal 50 is designed as an O-ring and is arranged in a plane that is inclined to the axis of rotation 5. By turning the seal carrier 30, the seal 50 arranged in the sealing groove 38 moves along the inside of the through-hole 12 and closes or opens the ventilation duct 14. The seal 50 is thus statically arranged on the pin 35 of the seal carrier 30 and is rotated together with the seal carrier 30 relative to the base body 10. In an alternative embodiment, the seal is arranged in a groove within the base body 10 and is statically mounted in the base body 10. The pin 35 with the channel 350 or the channels 350 rotates on the inside of the seal along its surface.

In FIG. 2, a sectional view of the ejection valve is shown in a different sectional plane, in which the sectional plane is shown perpendicular to the sectional plane of FIG. 1, which can be recognized by the same height of the sections of the seal 50. FIG. 2 also shows a groove 370 on the underside of the roof section of the seal carrier 30. A stop element or a stop is accommodated in this groove 370, which will be explained later. The groove 370 is designed as a positive locking element and serves as a mechanical limitation of the angle of rotation of the seal carrier 30 about the axis of rotation 5, so that the seal carrier 30 can only be rotated over a certain angle of rotation range.

FIG. 2 also shows the inwardly projecting projection 33 on the wall 31. In the illustrated embodiment example, the inner edge of the projection 33 borders on the outer circumference of the base body 10, with a small gap. In an alternative embodiment, the projection 33 can extend below the head section of the base body 10 and prevent axial displacement of the seal carrier 30 away from the base body 10. For this purpose, the lower edge of the seal carrier 30 is bent open laterally, moved over the base body 10 and then bent back, in particular due to elastic restoring forces. As an alternative to bending up, a web or a projection can be formed or arranged on the underside, which corresponds to a flattening or recess, so that the seal carrier can only be mounted on the base body in exactly one position, whereby the web or projection can be guided in a groove after mounting and the first twisting.

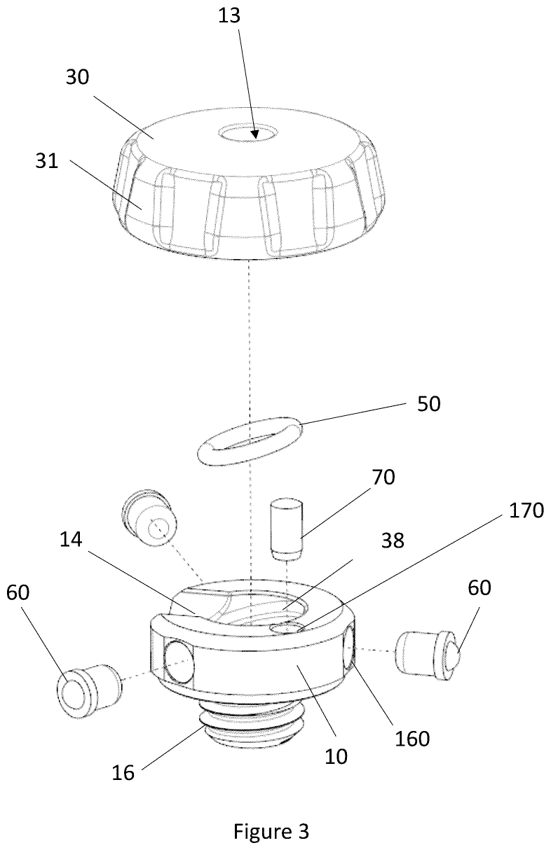

FIG. 3 shows an exploded view of the valve according to FIGS. 1 and 2 in a perspective view. The outlet duct 13 is formed inside the seal carrier 30, the wall 31 completely surrounds the roof area of the seal carrier 30. The inclined seal 50 can be seen. The inclined position of the seal 50 relative to the axis of rotation 5 results in the advantage of a low axial installation height with simultaneous displacement of the sealed area in the circumferential direction when the seal carrier 30 is rotated relative to the base body 10. The base body 10 with the external thread 16 at the lower end and the disc-shaped head area has three holes 160 on its outer circumference, which project radially inwards and serve to accommodate bearing bodies 60. The bearing bodies 60 are designed as a module and are inserted into the holes 160 for mounting. In an assembled state, the balls or spherical sections of the bearing bodies 60 project radially beyond the outer wall of the base body 10, the balls or spherical sections being resiliently mounted radially so that they can be moved into the bores 160 in the direction of the axis of rotation 5 against a spring force. After mounting the seal carrier 30 over the base body 10, the balls or spherical sections of the bearing bodies 60 are pressed outwards and pressed into the bearing groove, which is not shown.

A bore 170 oriented in the axial direction is formed in the upper side of the base body 10, into which a stop 70 is inserted, which protrudes beyond the upper side of the base body 10. The stop 70 can also be produced as part of a primary forming process, machined and formed in one piece or directly moulded on. The stop 70 engages in the groove 370, which is shown in FIG. 2, and limits the maximum angle of rotation of the seal carrier 30 relative to the base body 10. In the embodiment example shown in FIG. 3, the sealing groove 38 for the seal 50 is formed within the through-hole 12 in the base body 10. In this embodiment, the seal 50 is statically positioned within the base body 10.

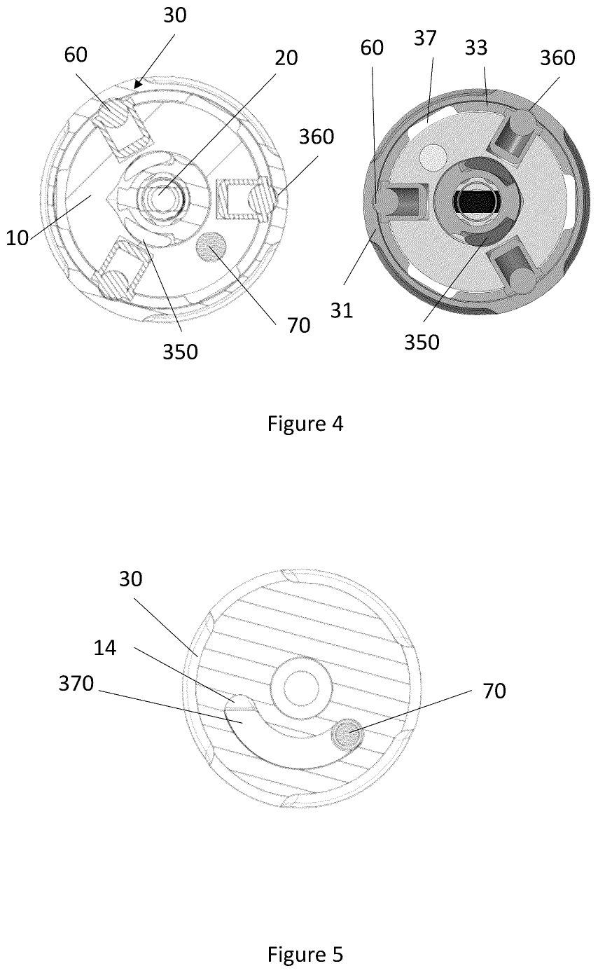

FIG. 4 shows horizontal sections through the socket valve in two different positions, which can be recognized by the position of the channels 350 and the stop 70. In the left-hand representation of FIG. 4, the ventilation duct 14 is open, while in the right-hand representation the ventilation duct, which is not shown, is closed. In the horizontal sectional view, recesses 37 can be seen within the projection 33. A total of three bearing bodies 60 are evenly distributed around the circumference of the base body 10. The bearing bodies 60 are resiliently mounted within bearing body carriers, which are inserted in the bores 160 as shown in FIG. 3. To simplify assembly, it is provided that the seal carrier 30 is aligned with the recesses 37 in line with the bearing bodies 60 and then moved downwards until the bearing bodies 60 engage in the bearing groove. The stop 70 is aligned in such a way that it engages in a recess or groove 370 that is arranged or formed on the seal carrier 30. The stop 70 advantageously limits the maximum angle of rotation to approximately 120°, so that when the stop 70 is correctly aligned with the groove 370, the bearing bodies 60 do not protrude above the recesses 37. This also prevents axial displacement of the bearing body carrier away from the base body 10. The positioning of the bearing body carrier with the stop 70 and the groove 370 formed as a circular arc section is shown in FIG. 5, in which a part of the ventilation duct 14 in the base body 10 can also be seen. In the illustrations in FIG. 4, detent recesses 360 for the bearing bodies 60 are shown within the side wall 31. The detent recesses 360 are each positioned in the end positions of the first position and the second position, so that there is an increased resistance to rotation about the axis of rotation 5. The bearing groove 36 can have a different radius over the circumference, whereby the radius is selected in the direction towards the end positions, in particular towards the closed end position, such that the bearing bodies 60 are maximally preloaded. In one configuration of the bearing groove 36 in the wall 31, the radius of the bearing groove 36 decreases towards the closed first position. With a reverse arrangement of the bearing groove within the base body 10, the radius increases towards the first position.

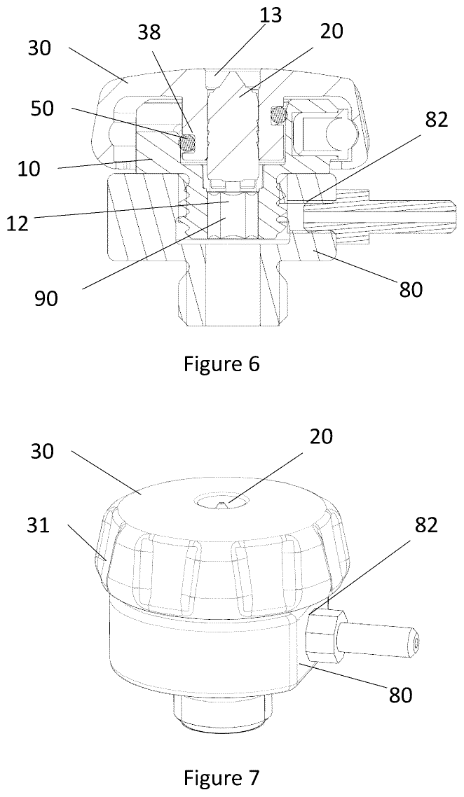

FIG. 6 shows a further variant of the ejection valve with the base body 10, the seal carrier 30 and the valve body 20. Instead of a spring-loaded valve body 20 movable in the axial direction along the axis of rotation 5 with a circumferential seal 62 for sealing in the valve seat 32, the valve body 20 is designed as a diaphragm valve or so-called beak valve, through which air can pass at any time at a corresponding differential pressure from the prosthesis socket through the through-hole 12 and the outlet duct 13 into the environment. Due to the material properties and the shape of the diaphragm valve, it closes at an inverse differential pressure or below an overpressure, so that no air can enter the prosthesis socket through the outlet duct 13. The seal 50 is again arranged in the sealing groove 38 on the pin 35 inside the seal carrier 30 and is oriented in an inclined plane to the axis of rotation 5 of the seal carrier 30. Via the external thread 16, the base body 10 is screwed into a base 80, on which a pump connection 82 is formed, in which a line is arranged. The pump connection 82 provides a fluidic connection with the through-opening 12 in the base body 10 and enables, for example, the suction of air via a pump before reaching the valve body 20. The base 80 in turn has fastening devices, for example an external thread, which can be screwed into a prosthesis socket in order to be able to actively suck air out of the prosthesis socket via the base 80 with the pump connection 82. The function of the socket valve as an ejection valve remains essentially unaffected by this. Instead of a cylindrical bore, an internal hexagon is formed within the through-opening 12 as a positive-locking element 90, with which it is possible to fasten the base body 10 to the base 80 or, in the case of direct mounting on a prosthesis socket, to a prosthesis socket using a corresponding hexagon key or Allen key.

FIG. 7 shows the discharge valve with the base 80 and a fluid line at the pump connection 82 in the assembled state. The valve body 20 is arranged centrally within the outlet duct 13.

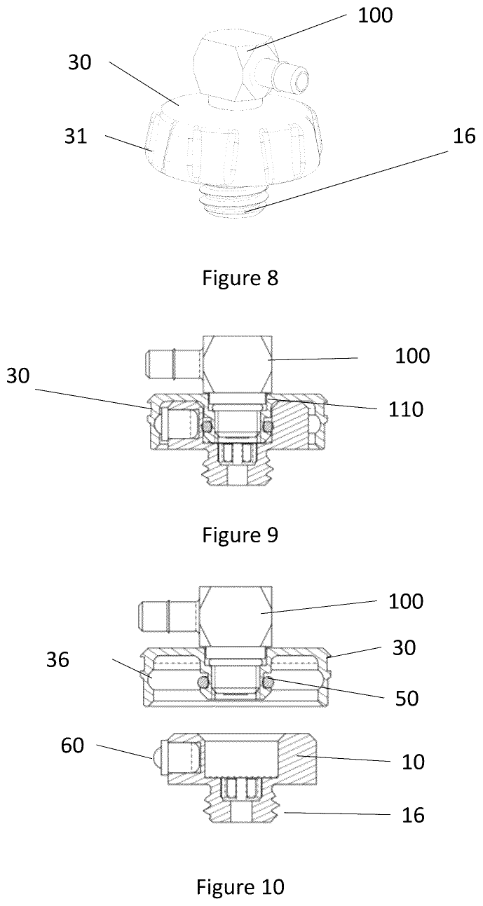

FIG. 8 again shows a perspective view of an ejection valve with the base body, of which the external thread 16 and the mounted seal carrier 30 with the wall 31 can be seen. A suction connection 100 is attached to the top of the seal carrier 30, with which it is possible to suck air out of the prosthesis socket through the seal carrier using a corresponding pump.

In the embodiments of FIGS. 8 to 10, no valve body is arranged in the seal carrier so that air can be sucked out or drained directly from the prosthesis shaft and does not have to flow through a non-return valve or past a spring-loaded valve body. If a valve body is arranged in the seal carrier, air can be actively extracted through the outlet duct due to the pump arranged behind the valve body in the direction of flow. This provides an ejection valve that can be actuated solely by the movement in the prosthesis socket in conjunction with active suction to provide a desired pressure level in the prosthesis socket.

FIG. 9 shows the mounted state of the suction connection 100 on the seal carrier 30. To secure the suction connection 100, fastening devices 110 are arranged or formed on the seal carrier 30, for example a thread, a carabiner fastener or a clamping device, via which the suction connection 100 can be fastened to the seal carrier 30 in as sealed a manner as possible. Advantageously, the suction connection 100 is detachably secured to the seal carrier 30.

FIG. 10 shows the sectional view according to FIG. 9 in a position that has not yet been assembled. Not all elements of the socket valve are shown in FIG. 10. It can be seen that the circumferential bearing groove 36 is formed within the side wall 31 of the seal carrier 30 and the suction connection 100 is arranged in the direction of flow behind the valve body 20 mounted in a variant in the seal carrier 30. For assembly, the seal carrier 30 is slid over the base body 10 and the bearing bodies 60 engage in the bearing groove 36. For easier assembly, recesses 37 may be formed in the inwardly directed projection 33, which here forms the lower groove wall.

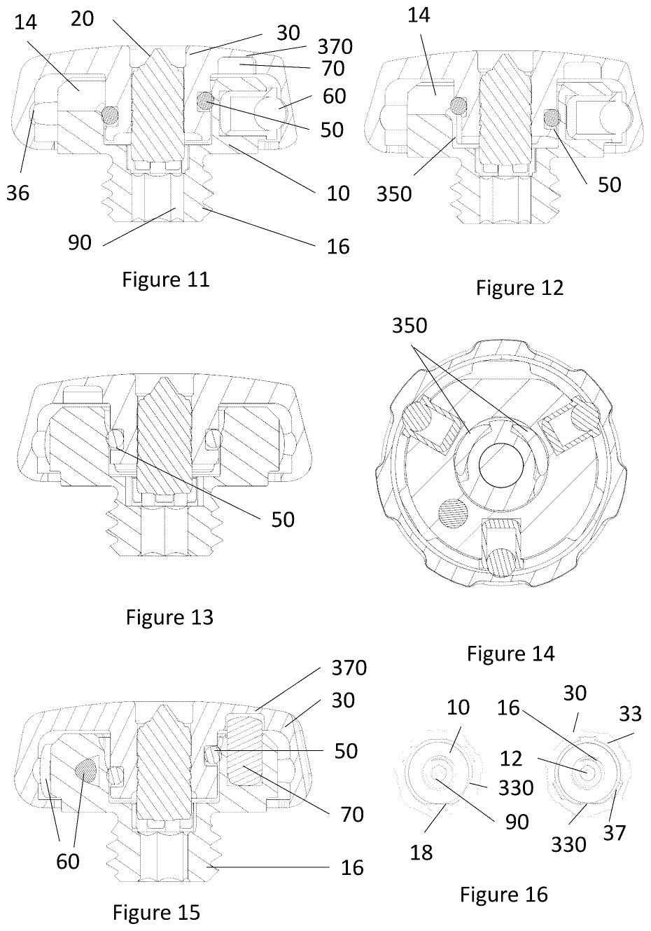

FIGS. 11 and 12 show the socket valve in the form of an ejection valve in sectional view with a valve body 20 as a diaphragm or beak valve, with a positive locking element 90 within the central through bore in the pin with the external thread 16 in the base body in the closed position in FIG. 11 and in the open position in FIG. 12. The stop 70 is guided within the groove 370 in the seal carrier 30. In FIG. 12, the seal carrier 30 is rotated by 120°. The stop is guided in the groove 370 up to the end of the groove, so that the channels 350 establish an unsealed fluidic connection between the through bore 12 in the base body 10 and the ventilation duct 14, which functions as a ventilation duct.

FIG. 13 shows the valve according to FIGS. 11 and 12 in a different section.

FIG. 14 shows a horizontal section with the two channels 350, which are formed in the pin 35 of the seal carrier 30. The bearing body 60, the detent recesses 360 and the stop 70 can also be seen.

FIG. 15 shows the stop 70 inserted in the base body 10 in the form of a bolt and the groove 370 in the seal carrier 30. Furthermore, a partially cut bearing body 60 can be seen in FIG. 15, the outside of which engages in the groove within the seal carrier 30. The mounting of the bearing body 60 in a recess within the body 10 and a correspondingly resilient mounting for applying a preload force acting radially outwards via a thrust piece ensures that the seal carrier 30 is securely attached to the base body 10.

FIG. 16 shows a bottom view of the socket valve in two positions with the underside of the base body 10, the form-locking element 90 within the through bore 12 and the seal carrier 30 with the inwardly projecting projection 33 and the recesses 37 formed therein. In the left-hand representation in FIG. 16, a flattening 18 can be seen in the base body 10. Likewise, a web or bridge 330 can be recognized as an inwardly projecting projection which, in the twisted state according to the left-hand representation of FIG. 16, overlaps with a circumferential web on the base body 10. In this position, the seal carrier 30 cannot move away from the base body 10 in the axial direction. Assembly is only possible in the position of the seal carrier 30 relative to the base body 10 as shown on the right in FIG. 16. Thus, assembly is only possible in an unambiguous assembly position, which is shown in the right-hand representation of FIG. 16. The web or bridge 330 is in overlap with the lateral flattening 18 and is congruent with it, so that the seal carrier 30 is placed on and joined by twisting in a final assembly.

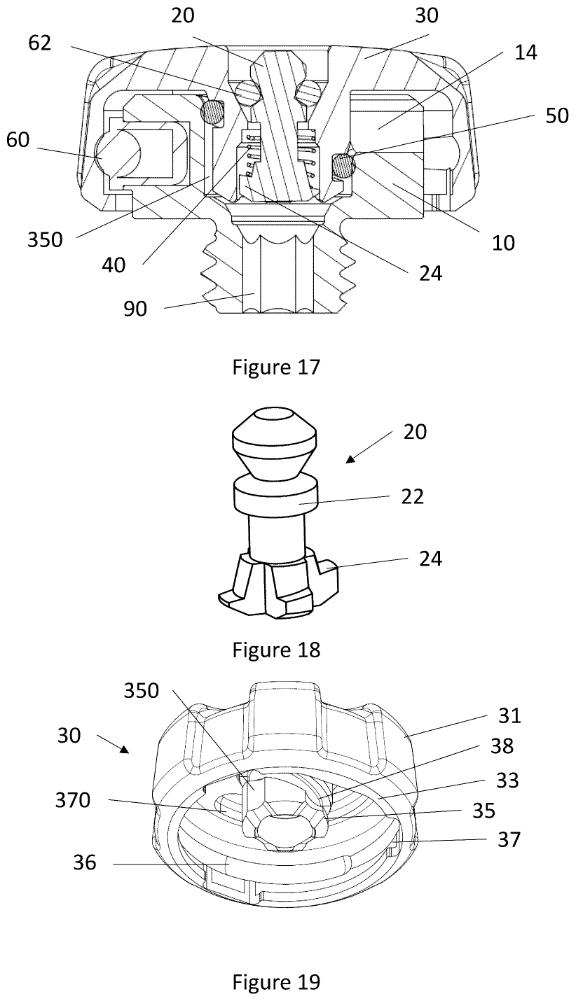

FIG. 17 shows a further embodiment of the ejection valve, whereby the basic structure corresponds to that of the embodiment described, for example, with reference to FIGS. 1 and 2. In addition to the positive-locking elements 90 in the through bore, a support 24 is formed on the valve body 20 at the lower end, which can be seen more clearly in FIG. 18, which shows a perspective overall view of the valve body 20. The support 24 supports the lower end of the valve spring 40 and ensures that the seal 62, which is arranged above the collar 22, is in sealing contact with the valve seat. The support 24 is formed by a total of four outwardly projecting shoulders, which in the embodiment shown are formed at different heights along the longitudinal extent of the valve body 20. This results in an oblique or inclined mounting of the valve spring 40, whereby the bearing surfaces of the support 24 can lie in a plane that is inclined to the longitudinal extent of the valve body 20. The resulting slight inclination of the valve spring 40 also causes an inclination of the valve body 20, which increases the friction when the valve body 20 is displaced. The increased friction prevents the valve body 20 from resonating and developing unpleasant noises at corresponding flow velocities of the escaping air. In order to avoid disruptive vibrations when the socket valve is designed as an ejection valve, an additional damping element is required for this spring-mass system, which is formed by the valve body 20 and the valve spring 40. The inclined support of the spring 40 on the support shoulders 24 of the valve body, which are arranged in an inclined plane, causes an additional transverse force on the valve body 20. The resulting inclined position and friction of the valve body on the side wall of the seal carrier 30 produces the desired damping behaviour.

FIG. 19 shows the underside of the seal carrier 30 with the inclined sealing groove 38 formed on the pin 35, as well as the projection 33 and the recesses 37 in the projection 33, which are evenly distributed around the circumference. The bearing groove 36 runs on a common plane and can have different radii. On the underside of the cover of the seal carrier 30, the groove 370 formed as an arc of a circle can be seen, as can the channels 350 oriented in the longitudinal direction of the pin 35.

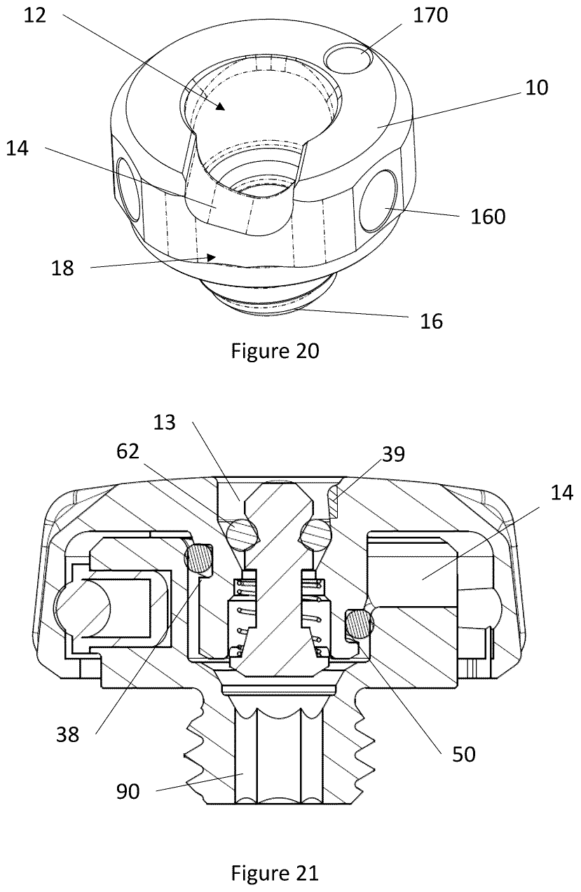

FIG. 20 shows an individual representation of the base body 10 with the holes 160 for the bearing bodies not shown, the hole 170 for the stop 70 and the ventilation duct 14. The pin 35 of the seal carrier 30 is inserted inside the through hole 12, which can have different diameters over the axial extension. In the area of the ventilation duct, the flattening 18 is formed on the outer side wall, which must be brought into overlap with the web 330 of the seal carrier 30 as shown in FIG. 16 in order to mount the seal carrier 30 on the base body 10.

FIG. 21 shows a further variant of the exhaust valve of FIG. 17, in which a substantially flat support for the valve spring is formed instead of an inclined or slanted support 24. In order to adapt or increase the friction, at least one contact section 39 is arranged or formed in the area of the upper end of the outlet duct 13, which projects radially inwards, instead of an inclined position of the spring. The contact section 39 can also be formed in one piece with the seal carrier 30 or produced during primary moulding.

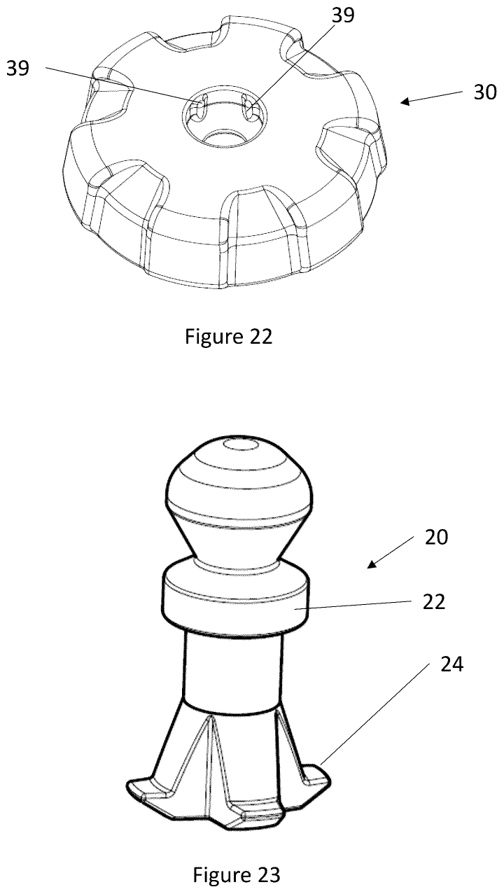

FIG. 22 shows two contact sections 39 that extend along the length of the outlet duct 13 and come into contact with the seal 62 when there is excess pressure in the prosthesis socket. FIG. 22 shows that the contact sections 39 extend as far as a radial taper of the outlet duct 13. In particular, they are arranged symmetrically around the circumference in order to achieve uniform guidance of the valve body 20. Due to the material composition and/or larger extension and/or the radial extension in the direction of the central axis of the through bore, different contact pressures and thus different frictions can be generated in order to achieve damping of the movement of the valve body 20.

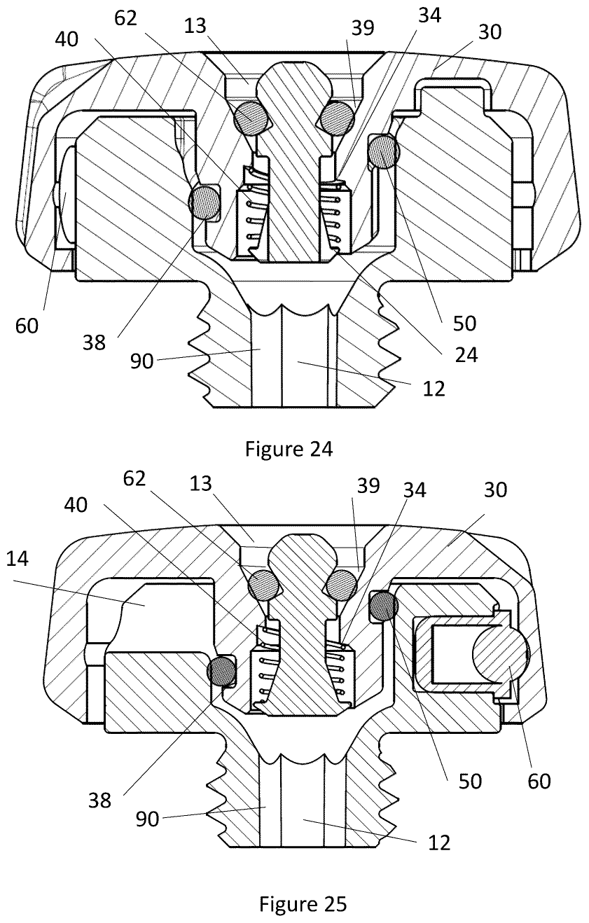

In FIG. 23, the valve body 20 is shown in a variant in which the shoulders forming the support 24 are arranged in a plane that is oriented essentially perpendicular to the longitudinal extension or longitudinal axis of the valve body 20. Such a valve body 20 is shown in the seal carrier 30 of FIG. 24 and FIG. 25. A spring seat 34 is formed within the seal carrier 30, which is formed in the shape of an inclined plane. The plane of the valve seat 32 is inclined at an angle to the longitudinal extent of the valve body 20 and is therefore not parallel to the plane of the shoulders that form the support 24. Due to the inclined position of the spring seat 34, a transverse force component is generated, similar to that explained in FIG. 18, in order to generate friction of the valve body 20 within the seal carrier 30 to provide a damping component in the system. This prevents disruptive vibrations in the ejection valve with the valve body 20.

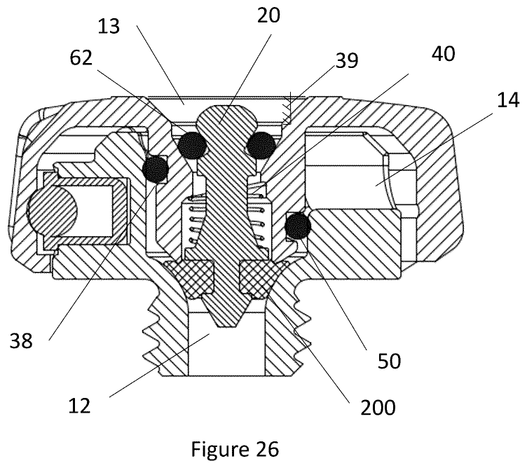

FIG. 26 shows a variant of a socket valve in a sectional view. The basic structure of the socket valve corresponds to the structure shown in FIG. 21. The valve body 20 in the embodiment shown in FIG. 26 is also resiliently mounted in the seal carrier with a circumferential seal 62 in the contact section of the valve seat, with the valve body 20 projecting beyond the pin and into the through bore 12 of the base body 10. At the lower end of the valve body 20 facing the base body 10, a silencer 200 is arranged below the outlet duct 13, the silencer is arranged circumferentially around the valve body 20. In the illustrated embodiment example, the silencer 200 is made of a foam material and inserted in a groove formed in the valve body 20. The silencer 200 extends around the entire circumference of the valve body 20 and rests with its outer circumference on the surface of the through bore 12 in the area below the seal carrier. The valve spring 40 is supported on the one hand on the valve body 20 and on the other hand on a projection within the through bore 12, whereby the contact surface or the projection within the through bore 12 is inclined so that a transverse force component is generated in order to provide a damping component in the system and to prevent vibrations of the valve body 20. The air flows through the silencer 200 in order to reduce noise when air is released during opening and when air is expelled in the event of overpressure. The silencer 200 is designed, for example, in such a way that it dampens the air passage even when the valve body 20 is raised by expanding.

### LIST OF REFERENCE SYMBOLS

5—Rotary axis
10—Base body
12—Through hole
13—Outlet duct
14—Ventilation duct
16—External thread
18—Flattening
20—Valve body
22—Collar
24—Support
30—Seal carrier
31—Wall
32—Valve seat
33—Projection
34—Spring seat
35—Pin
36—Bearing groove
37—Recess
38—Sealing groove
39—Contact section
40—Valve spring
50—Seal
60—Bearing body
62—Seal
70—Stop
80—Base
82—Pump connection
90—Positive locking element
100—Suction connection
110—Fastening device
160—Bore
170—Bore
200—Silencer
330—Bridge
350—Channel
360—Detent recess
370—Groove

We claim:

1. Socket valve for ventilating and venting a prosthesis socket with a base body (10), the base body (10) has a through bore (12), a seal carrier (30) mounted on the base body (10) so as to be rotatable about an axis of rotation (5) is attached to the base body (10), which is in fluidic connection with the through-bore (12) and closes or opens a ventilation duct (14) which is in fluidic connection with the through-bore (12), characterized in in that the closable ventilation duct (14) is formed in the base body (10) and is sealed off from the through bore (12) via a seal (50) arranged between the seal carrier (30) and the base body (10) in a first position of the seal carrier (30) and is in fluidic connection with the through bore (12) in an unsealed manner in a second position of the seal carrier (30), and the seal (50) is positioned in an inclined manner with respect to the axis of rotation (5).

2. Socket valve according to claim 1, characterized in that an outlet duct (13) is formed in the seal carrier (30), which is in fluidic connection with the through bore (12) and in which a valve body (20) is arranged.

3. Socket valve according to claim 2, characterized in that the valve body (20) closes the outlet duct (13) with respect to a differential pressure.

4. Socket valve according to claim 2, characterized in that the valve body (20) is arranged in a valve seat (32) and is held prestressed in a closed position via a valve spring (40) or is designed as a beak valve.

5. Socket valve according to claim 2, characterized in that a seal (62) is arranged between the valve body (20) and the valve seat (32).

6. Socket valve according to claim 1, characterized in that the seal carrier (30) is mounted on the base body (10) via at least one bearing body (60), which runs along in a bearing groove (36).

7. Socket valve according to claim 6, characterized in that the bearing body (60) is resiliently mounted in the base body (10) and the bearing groove (36) is formed in the seal carrier (30) or vice versa.

8. Socket valve according to claim 6, characterized in that at least one detent recess (360) for the bearing body (60) is formed in the bearing groove (36).

9. Socket valve according to claim 1, characterized in that the seal carrier (30) has a wall (31) surrounding the base body (10) at least partially on the outside and having a radially inwardly projecting projection (33).

10. Socket valve according to claim 9, characterized in that at least one recess (37) is formed in the projection (33).

11. Socket valve according to claim 6, characterized in that the number of bearing bodies (60) corresponds to the number of recesses (37) and/or in that the size of the bearing bodies (60) corresponds to the size of the recesses (37).

12. Socket valve according to claim 1, characterized in that a stop (70) for limiting the angle of rotation is formed between the base body (10) and the seal carrier (30).

13. Socket valve according to claim 1, characterized in that an external thread (16) surrounding the through bore (12) is formed on the base body (10).

14. Socket valve according to claim 1, characterized in that the base body (10) has a positive-locking element (90) for engaging with an assembly tool.

15. Socket valve according to claim 1, characterized in that the base body (10) is inserted in a base (80) with a pump connection (82).

16. Socket valve according to claim 1, characterized in that the seal (50) is mounted on the outside of a pin (35) of the seal carrier (30) or on the inside of the base body (10) in a sealing groove (38).

17. Socket valve according to claim 1, characterized in that a fastening device (110) for a suction connection (100) is arranged on the seal carrier (30)

18. Socket valve according to claim 1, characterized in that the suction connection (100) is arranged downstream of the valve body (20) in the direction of flow.

19. Socket valve according to claim 1, characterized in that the seal carrier (30) is mounted on the base body (10) without axial stroke along the axis of rotation (5).

20. Socket valve according to claim 1, characterized in that a silencer (200) is arranged between the valve body (20) and the through bore (12).
